(19) United States
(12) Patent Application Publication
(10) Pub. No.: US 2005/0106166 A1
(43) Pub. Date: May 19, 2005

(54) Methods and compositions for treating liver cirrhosis

(76) Inventor: Ling Yuk Cheung, New Territories (HK)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/717,008

(22) Filed: Nov. 18, 2003

(51) Int. Cl. A61K 35/70; A61K 35/72

(52) U.S. Cl. 424/195.16

(57) ABSTRACT

The invention provides compositions comprising a plurality of yeast cells, wherein said plurality of yeast cells are characterized by their ability to decrease liver collagen level or formation of liver fibrous tissue or to normalize serum γ-globulin level, and therefore are useful for ameliorating and/or preventing liver cirrhosis in a subject, said ability resulting from their having been cultured in the presence of an alternating electric field having a specific frequency and a specific field strength. Also provided are methods of making and using these compositions.

### FIELD OF THE INVENTION

The invention relates to yeast compositions that can ameliorate or prevent liver cirrhosis and are useful as a dietary supplement or medication. These compositions contain yeast cells obtainable by growth in electromagnetic fields with specific frequencies and field strengths.

### BACKGROUND OF THE INVENTION

Liver cirrhosis, or cirrhosis, is a chronic liver disease in which fibrous tissue and nodules replace normal tissue, interfering with blood flow and normal functions of the organ. Cirrhosis can be caused by, e.g., chronic alcoholism, chronic viral hepatitis (types B, C, and D), cystic fibrosis, severe reactions to prescribed drugs, prolonged exposure to environmental toxins, etc.

Cirrhosis causes irreversible liver damage. If untreated, liver and kidney failure and gastrointestinal hemorrhage can occur, sometimes leading to death. In the United States, cirrhosis results in about 25,000 deaths annually. Apart from a vegetable protein-rich diet, abstinence from alcohol and rest, common medication includes vitamin B, vitamin E, vitamin C, etc. But these treatments are less than satisfactory. There remains a need for an effective method for treating liver cirrhosis.

### SUMMARY OF THE INVENTION

This invention is based on the discovery that certain yeast cells can be activated by electromagnetic fields having specific frequencies and field strengths to produce substances beneficial for the liver and therefore improving liver health. Compositions comprising these activated yeast cells can be used as dietary supplement for alleviating and/or preventing liver cirrhosis.

This invention embraces a composition comprising a plurality of yeast cells that have been cultured in an alternating electric field having a frequency in the range of about 7700-12800 MHz (e.g., 7800-8000 or 12150-12750 MHz), and a field intensity in the range of about 240-500 mV/cm (e.g., 260-280, 270-290, 300-330, 310-340, 320-350, 330-370, 340-370, 350-380, 400-440, or 430-470 mV/cm). The yeast cells are cultured in the alternating electric field for a period of time sufficient to substantially increase the capability of said plurality of yeast cells to produce substances beneficial for the liver (e.g., for treating cirrhosis). In one embodiment, the frequency and/or the field strength of the alternating electric field can be altered within the aforementioned ranges during said period of time. In other words, the yeast cells can be exposed to a series of electromagnetic fields. An exemplary period of time is about 40-160 hours (e.g., 60-150 hours).

Also included in this invention is a composition comprising a plurality of yeast cells that have been cultured under acidic conditions in an alternating electric field having a frequency in the range of about 12150-12750 MHz (e.g., 12550-12750 MHz) and a field strength in the range of about 280 to 420 mV/cm (e.g., 320-380 mV/cm). In one embodiment, the yeast cells are exposed to a series of electromagnetic fields. An exemplary period of time is about 30-100 hours (e.g., 40-74 hours).

Included in this invention are also methods for making the above compositions.

Yeast cells that can be included in this composition can be derived from parent strains publically available from the China General Microbiological Culture Collection Center (“CGMCC”), China Committee for Culture Collection of Microorganisms, Institute of Microbiology, Chinese Academy of Sciences, Haidian, P.O. BOX 2714, Beijing, 100080, China. Useful yeast species include, but are not limited to Saccharomyces cerevisiae, Saccharomyces carlsbergensis, Saccharomyces rouxii, Saccharomyces sake, Saccharomyces uvarum, Saccharomyces sp., Schizosaccharomyces pombe, and Rhodotorula aurantiaca. For instance, the yeast cells can be of the strain Saccharomyces cerevisiae Hansen AS2.562 or AS2.69, Saccharomyces sp. AS2.311, Schizosaccharomyces pombe Lindner AS2.994, Saccharomyces sake Yabe ACCC2045, Saccharomyces uvarum Beijer IFFI1044, Saccharomyces rouxii Boutroux AS2.180, Saccharomyces cerevisiae Hansen Var. ellipsoideus AS2.612, Saccharomyces carlsbergensis Hansen AS2.377, or Rhodotorula rubar (Demme) Lodder AS2.282. Other useful yeast strains are illustrated in Table 1.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. The materials, methods, and examples are illustrative only and not intended to be limiting. Throughout this specification and claims, the word “comprise,” or variations such as “comprises” or “comprising” will be understood to imply the inclusion of a stated integer or group of integers but not the exclusion of any other integer or group of integers.

Other features and advantages of the invention will be apparent from the following detailed description, and from the claims.

### BRIEF DESCRIPTION OF THE DRAWINGS

FIG. 1 is a schematic diagram showing an exemplary apparatus for activating yeast cells using electromagnetic fields. 1: yeast culture; 2: container; 3: power supply.

FIG. 2 is a schematic diagram showing an exemplary apparatus for making yeast compositions of the invention. The apparatus comprises a signal generator (such as models 83721B and 83741A manufactured by HP) and interconnected containers A, B and C.

### DETAILED DESCRIPTION OF THE INVENTION

This invention is based on the discovery that certain yeast strains can be activated by electromagnetic fields (“EMF”) having specific frequencies and field strengths to produce agents useful for treating liver cirrhosis. Yeast compositions containing activated yeast cells can be used as medication, or as a dietary supplement in the form of health drinks or dietary pills.

In certain embodiments, the yeast compositions of this invention inhibit the synthesis and secretion of collagen in liver. In other embodiments, the yeast compositions inhibit the formation of intra- and inter-molecular cross-linking of collagen molecules. In further embodiments, the yeast compositions reduce the level of serum γ-globulin.

Since the activated yeast cells contained in these yeast compositions have been cultured to endure acidic conditions (pH 2.5-4.2), the compositions are stable in the stomach and can pass on to the intestines. Once in the intestines, the yeast cells are ruptured by various digestive enzymes, and the bioactive agents are released and readily absorbed.

I. Yeast Strains Useful in the Invention

The types of yeasts useful in this invention include, but are not limited to, yeasts of the genera of Saccharomyces, Rhodotorula, and Schizosaccharomyces.

Exemplary species within the above-listed genera include, but are not limited to, the species illustrated in Table 1. Yeast strains useful in this invention can be obtained from laboratory cultures, or from publically accessible culture depositories, such as CGMCC and the American Type Culture Collection, 10801 University Boulevard, Manassas, Va. 20110-2209. Non-limiting examples of useful strains (with the accession numbers of CGMCC) are Saccharomyces cerevisiae Hansen AS2.562 and AS2.69, Saccharomyces sp. AS2.311, Schizosaccharomyces pombe Lindner AS2.994, Saccharomyces sake Yabe ACCC2045, Saccharomyces uvarum Beijer IFFI1044, Saccharomyces rouxii Boutroux AS2.180, Saccharomyces cerevisiae Hansen Var. ellipsoideus AS2.612, Saccharomyces carlsbergensis Hansen AS2.377, and Rhodotorula rubar (Demme) Lodder AS2.282. Other non-limiting examples of useful strains are listed in Table 1. In general, preferred yeast strains in this invention are those used for fermentation in the food and wine industries. As a result, compositions containing these yeast cells are safe for human consumption.

The preparation of the yeast compositions of this invention is not limited to starting with a pure strain of yeast. A yeast composition of the invention may be produced by culturing a mixture of yeast cells of different species or strains.

TABLE 1
Exemplary Yeast Strains
Saccharomyces cerevisiae Hansen

| | | | | |
|---|---|---|---|---|
| ACCC2034 | ACCC2035 | ACCC2036 | ACCC2037 | ACCC2038 |
| ACCC2039 | ACCC2040 | ACCC2041 | ACCC2042 | AS2.1 |
| AS2.4 | AS2.11 | AS2.14 | AS2.16 | AS2.56 |
| AS2.69 | AS2.70 | AS2.93 | AS2.98 | AS2.101 |
| AS2.109 | AS2.110 | AS2.112 | AS2.139 | AS2.173 |
| AS2.174 | AS2.182 | AS2.196 | AS2.242 | AS2.336 |
| AS2.346 | AS2.369 | AS2.374 | AS2.375 | AS2.379 |
| AS2.380 | AS2.382 | AS2.390 | AS2.393 | AS2.395 |
| AS2.396 | AS2.397 | AS2.398 | AS2.399 | AS2.400 |
| AS2.406 | AS2.408 | AS2.409 | AS2.413 | AS2.414 |
| AS2.415 | AS2.416 | AS2.422 | AS2.423 | AS2.430 |
| AS2.431 | AS2.432 | AS2.451 | AS2.452 | AS2.453 |
| AS2.458 | AS2.460 | AS2.463 | AS2.467 | AS2.486 |
| AS2.501 | AS2.502 | AS2.503 | AS2.504 | AS2.516 |
| AS2.535 | AS2.536 | AS2.558 | AS2.560 | AS2.561 |
| AS2.562 | AS2.576 | AS2.593 | AS2.594 | AS2.614 |
| AS2.620 | AS2.628 | AS2.631 | AS2.666 | AS2.982 |
| AS2.1190 | AS2.1364 | AS2.1396 | IFFI1001 | IFFI1002 |
| IFFI1005 | IFFI1006 | IFFI1008 | IFFI1009 | IFFI1010 |
| IFFI1012 | IFFI1021 | IFFI1027 | IFFI1037 | IFFI1042 |
| IFFI1043 | IFFI1045 | IFFI1048 | IFFI1049 | IFFI1050 |
| IFFI1052 | IFFI1059 | IFFI1060 | IFFI1062 | IFFI1063 |
| IFFI1202 | IFFI1203 | IFFI1206 | IFFI1209 | IFFI1210 |
| IFFI1211 | IFFI1212 | IFFI1213 | IFFI1214 | IFFI1215 |
| IFFI1220 | IFFI1221 | IFFI1224 | IFFI1247 | IFFI1248 |
| IFFI1251 | IFFI1270 | IFFI1277 | IFFI1287 | IFFI1289 |
| IFFI1290 | IFFI1291 | IFFI1292 | IFFI1293 | IFFI1297 |
| IFFI1300 | IFFI1301 | IFFI1302 | IFFI1307 | IFFI1308 |
| IFFI1309 | IFFI1310 | IFFI1311 | IFFI1331 | IFFI1335 |
| IFFI1336 | IFFI1337 | IFFI1338 | IFFI1339 | IFFI1340 |
| IFFI1345 | IFFI1348 | IFFI1396 | IFFI1397 | IFFI1399 |
| IFFI1411 | IFFI1413 | IFFI1441 | IFFI1443 | |

Saccharomyces cerevisiae Hansen Var. ellipsoideus (Hansen) Dekker

| | | | | |
|---|---|---|---|---|
| ACCC2043 | AS2.2 | AS2.3 | AS2.8 | AS2.53 |
| AS2.163 | AS2.168 | AS2.483 | AS2.541 | AS2.559 |
| AS2.606 | AS2.607 | AS2.611 | AS2.612 | |

Saccharomyces chevalieri Guilliermond

| | |
|---|---|
| AS2.131 | AS2.213 |

Saccharomyces delbrueckii

AS2.285

Saccharomyces delbrueckii Lindner ver. mongolicus (Saito) Lodder et van Rij

| | |
|---|---|
| AS2.209 | AS2.1157 |

Saccharomyces exiguous Hansen

| | |
|---|---|
| AS2.349 | AS2.1158 |

Saccharomyces fermentati (Saito) Lodder et van Rij

| | |
|---|---|
| AS2.286 | AS2.343 |

Saccharomyces logos van laer et Denamur ex Jorgensen

| | | |
|---|---|---|
| AS2.156 | AS2.327 | AS2.335 |

Saccharomyces mellis (Fabian et Quinet) Lodder et kreger van Rij

AS2.195

Saccharomyces mellis Microellipsoides Osterwalder

AS2.699

Saccharomyces oviformis Osteralder

AS2.100

Saccharomyces rosei (Guilliermond) Lodder et Kreger van Rij

AS2.287

Saccharomyces rouxii Boutroux

| | | | |
|---|---|---|---|
| AS2.178 | AS2.180 | AS2.370 | AS2.371 |

Saccharomyces sake Yabe

ACCC2045

Candida arborea

AS2.566

Candida lambica (Lindner et Genoud) van. Uden et Buckley

AS2.1182

Candida krusei (Castellani) Berkhout

AS2.1045

Candida lipolytica (Harrison) Diddens et Lodder

| | | | | |
|---|---|---|---|---|
| AS2.1207 | AS2.1216 | AS2.1220 | AS2.1379 | AS2.1398 |
| AS2.1399 | AS2.1400 | | | |

Candida parapsilosis (Ashford) Langeron et Talice Var. intermedia Van Rij et Verona

AS2.491

Candida parapsilosis (Ashford) Langeron et Talice

AS2.590

Candida pulcherrima (Lindner) Windisch

AS2.492

Candida rugousa (Anderson) Diddens et Lodder

| | | | | |
|---|---|---|---|---|
| AS2.511 | AS2.1367 | AS2.1369 | AS2.1372 | AS2.1373 |
| AS2.1377 | AS2.1378 | AS2.1384 | | |

Candida tropicalis (Castellani) Berkhout

| | | | | |
|---|---|---|---|---|
| ACCC2004 | ACCC2005 | ACCC2006 | AS2.164 | AS2.402 |
| AS2.564 | AS2.565 | AS2.567 | AS2.568 | AS2.617 |
| AS2.637 | AS2.1387 | AS2.1397 | | |

Candida utilis Henneberg Lodder et Kreger Van Rij

| | | |
|---|---|---|
| AS2.120 | AS2.281 | AS2.1180 |

Crebrothecium ashbyii (Guillermond)
Routein (Eremothecium ashbyii Guilliermond)

| | | |
|---|---|---|
| AS2.481 | AS2.482 | AS2.1197 |

Geotrichum candidum Link

| | | | | |
|---|---|---|---|---|
| ACCC2016 | AS2.361 | AS2.498 | AS2.616 | AS2.1035 |
| AS2.1062 | AS2.1080 | AS2.1132 | AS2.1175 | AS2.1183 |

Hansenula anomala (Hansen)H et P sydow

| | | | | |
|---|---|---|---|---|
| ACCC2018 | AS2.294 | AS2.295 | AS2.296 | AS2.297 |
| AS2.298 | AS2.299 | AS2.300 | AS2.302 | AS2.338 |
| AS2.339 | AS2.340 | AS2.341 | AS2.470 | AS2.592 |
| AS2.641 | AS2.642 | AS2.782 | AS2.635 | AS2.794 |

Hansenula arabitolgens Fang

AS2.887

Hansenula jadinii (A. et R Sartory Weill et Meyer) Wickerham

ACCC2019

Hansenula saturnus (Klocker) H et P sydow

ACCC2020

Hansenula schneggii (Weber) Dekker

AS2.304

Hansenula subpelliculosa Bedford

| | | | | |
|---|---|---|---|---|
| AS2.740 | AS2.760 | AS2.761 | AS2.770 | AS2.783 |
| AS2.790 | AS2.798 | AS2.866 | | |

Kloeckera apiculata (Reess emend. Klocker) Janke

| | | | | |
|---|---|---|---|---|
| ACCC2022 | ACCC2023 | AS2.197 | AS2.496 | AS2.714 |
| ACCC2021 | AS2.711 | | | |

Lipomycess starkeyi Lodder et van Rij

| | |
|---|---|
| AS2.1390 | ACCC2024 |

Pichia farinosa (Lindner) Hansen

| | | | | |
|---|---|---|---|---|
| ACCC2025 | ACCC2026 | AS2.86 | AS2.87 | AS2.705 |
| AS2.803 | | | | |

Pichia membranaefaciens Hansen

| | | | |
|---|---|---|---|
| ACCC2027 | AS2.89 | AS2.661 | AS2.1039 |

Rhodosporidium toruloides Banno

ACCC2028

Rhodotorula glutinis (Fresenius) Harrison

| | | | | |
|---|---|---|---|---|
| AS2.2029 | AS2.280 | ACCC2030 | AS2.102 | AS2.107 |
| AS2.278 | AS2.499 | AS2.694 | AS2.703 | AS2.704 |
| AS2.1146 | | | | |

Rhodotorula minuta (Saito) Harrison

AS2.277

Rhodotorula rubar (Demme) Lodder

| | | | | |
|---|---|---|---|---|
| AS2.21 | AS2.22 | AS2.103 | AS2.105 | AS2.108 |
| AS2.140 | AS2.166 | AS2.167 | AS2.272 | AS2.279 |
| AS2.282 | ACCC2031 | | | |

Rhodotorula aurantiaca (Saito) Lodder

| | | | | |
|---|---|---|---|---|
| AS2.102 | AS2.107 | AS2.278 | AS2.499 | AS2.694 |
| AS2.703 | AS2.1146 | | | |

Saccharomyces carlsbergensis Hansen

| | | | | |
|---|---|---|---|---|
| AS2.113 | ACCC2032 | ACCC2033 | AS2.312 | AS2.116 |
| AS2.118 | AS2.121 | AS2.132 | AS2.162 | AS2.189 |

Saccharomyces uvarum Beijer

| | | | | |
|---|---|---|---|---|
| IFFI1023 | IFFI1032 | IFFI1036 | IFFI1044 | IFFI1072 |
| IFFI1205 | IFFI1207 | | | |

Saccharomyces willianus Saccardo

| | | | | |
|---|---|---|---|---|
| AS2.5 AS2.7 | AS2.119 | AS2.152 | AS2.293 | |
| AS2.381 | AS2.392 | AS2.434 | AS2.614 | AS2.1189 |

Saccharomyces sp.

AS2.311

Saccharomycodes ludwigii Hansen

| | | |
|---|---|---|
| ACCC2044 | AS2.243 | AS2.508 |

Saccharomycodes sinenses Yue

AS2.1395

Schizosaccharomyces octosporus Beijerinck

| | |
|---|---|
| ACCC2046 | AS2.1148 |

Schizosaccharomyces pombe Lindner

| | | | | |
|---|---|---|---|---|
| ACCC2047 | ACCC2048 | AS2.214 | AS2.248 | AS2.249 |
| AS2.255 | AS2.257 | AS2.259 | AS2.260 | AS2.274 |
| AS2.994 | AS2.1043 | AS2.1149 | AS2.1178 | IFFI1056 |

Sporobolomyces roseus Kluyver et van Niel

| | | | | |
|---|---|---|---|---|
| ACCC2049 | ACCC2050 | AS2.19 | AS2.962 | AS2.1036 |
| ACCC2051 | AS2.261 | AS2.262 | | |

Torulopsis candida (Saito) Lodder

| | |
|---|---|
| AS2.270 | ACCC2052 |

Torulopsis famta (Harrison) Lodder et van Rij

| | |
|---|---|
| ACCC2053 | AS2.685 |

Torulopsis globosa (Olson et Hammer) Lodder et van Rij

| | |
|---|---|
| ACCC2054 | AS2.202 |

Torulopsis inconspicua Lodder et Kreger van Rij

AS2.75

Trichosporon behrendii Lodder et Kreger van Rij

| | |
|---|---|
| ACCC2056 | AS2.1193 |

Trichosporon capitatum Diddens et Lodder

| | |
|---|---|
| ACCC2056 | AS2.1385 |

Trichosporon cutaneum (de Beurm et al.) Ota

| | | | | |
|---|---|---|---|---|
| ACCC2057 | AS2.25 | AS2.570 | AS2.571 | AS2.1374 |

Wickerhamia fluorescens (Soneda) Soneda

| | |
|---|---|
| ACCC2058 | AS2.1388 |

II. Application of Electromagnetic Fields

An electromagnetic field useful in this invention can be generated and applied by various means well known in the art. For instance, the EMF can be generated by applying an alternating electric field or an oscillating magnetic field.

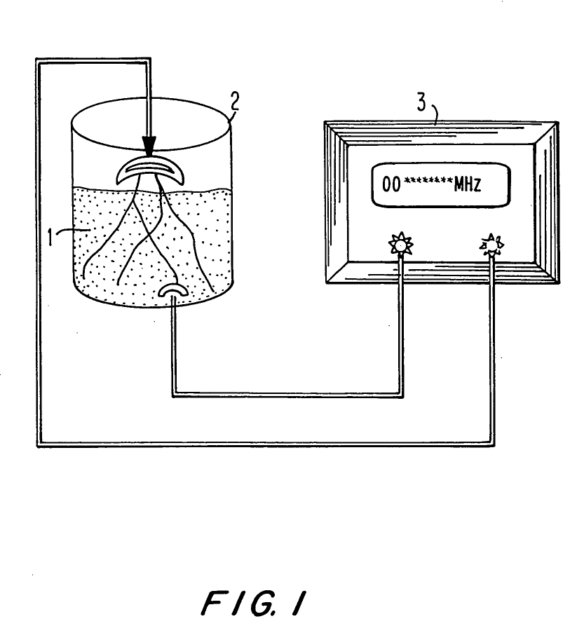

Alternating electric fields can be applied to cell cultures through electrodes in direct contact with the culture medium, or through electromagnetic induction. See, e.g., FIG. 1. Relatively high electric fields in the medium can be generated using a method in which the electrodes are in contact with the medium. Care must be taken to prevent electrolysis at the electrodes from introducing undesired ions into the culture and to prevent contact resistance, bubbles, or other features of electrolysis from dropping the field level below that intended. Electrodes should be matched to their environment, for example, using Ag—AgCl electrodes in solutions rich in chloride ions, and run at as low a voltage as possible. For general review, see Goodman et al., Effects of EMF on Molecules and Cells, International Review of Cytology, A Survey of Cell Biology, Vol. 158, Academic Press, 1995.

The EMFs useful in this invention can also be generated by applying an oscillating magnetic field. An oscillating magnetic field can be generated by oscillating electric currents going through Helmholtz coils. Such a magnetic field in turn induces an electric field.

The frequencies of EMFs useful in this invention range from about 7700-12800 MHz (e.g., 7800-8000 or 12150-12750 MHz). Exemplary frequencies include 7886, 7907, 12224, 12646, and 12662 MHz. The field strength of the electric field useful in this invention ranges from about 240-500 mV/cm (e.g., 260-280, 270-290, 300-330, 310-340, 320-350, 330-370, 340-370, 350-380, 400-440, or 430-470 mV/cm). Exemplary field strengths include 274, 278, 311, 324, 337, 347, 355, 364, 368, 413, and 442 mV/cm.

When a series of EMFs are applied to a yeast culture, the yeast culture can remain in the same container while the same set of EMF generator and emitters is used to change the frequency and/or field strength. The EMFs in the series can each have a different frequency or a different field strength; or a different frequency and a different field strength. Such frequencies and field strengths are preferably within the above-described ranges. Although any practical number of EMFs can be used in a series, it may be preferred that the yeast culture be exposed to a total of 2, 3, 4, 5, 6, 7, 8, 9, 10, 11, 12, 13, or more EMFs in a series. In one embodiment, the yeast culture is exposed to a series of EMFs, wherein the frequency of the electric field is alternated in the range of about 7800-8000, 12150-12300, and 12550-12800 MHz.

Although the yeast cells can be activated after even a few hours of culturing in the presence of an EMF, it may be preferred that the activated yeast cells be allowed to multiply and grow in the presence of the EMF(s) for a total of 40-160 hours.

FIG. 1 illustrates an exemplary apparatus for generating alternating electric fields. An electric field of a desired frequency and intensity can be generated by an AC source (3) capable of generating an alternating electric field, preferably in a sinusoidal wave form, in the frequency range of 5 to 20,000 MHz. Signal generators capable of generating signals with a narrower frequency range can also be used. If desired, a signal amplifier can also be used to increase the output. The culture container (2) can be made from a non-conductive material, e.g., glass, plastic or ceramic. The cable connecting the culture container (2) and the signal generator (3) is preferably a high frequency coaxial cable with a transmission frequency of at least 30 GHz.

The alternating electric field can be applied to the culture by a variety of means, including placing the yeast culture (1) in close proximity to the signal emitters such as a metal wire or tube capable of transmitting EMFs. The metal wire or tube can be made of red copper, and be placed inside the container (2), reaching as deep as 3-30 cm. For example, if the fluid in the container (2) has a depth of 15-20 cm, 20-30 cm, 30-50 cm, 50-70 cm, 70-100 cm, 100-150 cm or 150-200 cm, the metal wire can be 3-5 cm, 5-7 cm, 7-10 cm, 10-15 cm, 15-20 cm, 20-30 cm, and 25-30 cm from the bottom of the container (2), respectively. The number of metal wires/tubes used can be from 1 to 10 (e.g., 2 to 3). It is recommended, though not mandated, that for a culture having a volume up to 10 L, metal wires/tubes having a diameter of 0.5 to 2 mm be used. For a culture having a volume of 10-100 L, metal wires/tubes having a diameter of 3 to 5 mm can be used. For a culture having a volume of 100-1000 L, metal wires/tubes having a diameter of 6 to 15 mm can be used. For a culture having a volume greater than 1000 L, metal wires/tubes having a diameter of 20-25 mm can be used.

In one embodiment, the electric field is applied by electrodes submerged in the culture (1). In this embodiment, one of the electrodes can be a metal plate placed on the bottom of the container (2), and the other electrode can comprise a plurality of electrode wires evenly distributed in the culture (1) so as to achieve even distribution of the electric field energy.

III. Culture Media

Culture media useful in this invention contain sources of nutrients that can be assimilated by yeast cells. Complex carbon-containing substances in a suitable form (e.g., carbohydrates such as sucrose, glucose, dextrose, maltose, xylose, cellulose, starch, etc.) can be the carbon sources for yeast cells. The exact quantity of the carbon sources can be adjusted in accordance with the other ingredients of the medium. In general, the amount of carbohydrate varies between about 1% and 10% by weight of the medium and preferably between about 1% and 5%, and most preferably about 2%. These carbon sources can be used individually or in combination. Amino acid-containing substances such as beef extract and peptone can also be added. In general, the amount of amino acid containing substances varies between about 0.1% and 1% by weight of the medium and preferably between about 0.1% and 0.5%. Among the inorganic salts which can be added to a culture medium are the customary salts capable of yielding sodium, potassium, calcium, phosphate, sulfate, carbonate, and like ions. Non-limiting examples of nutrient inorganic salts are (NH₄)₂HPO₄, CaCO₃, KH₂PO₄, K₂ HPO₄, MgSO₄, NaCl, and CaSO₄.

IV. Electromagnetic Activation of Yeast Cells

To activate or enhance the ability of yeast cells to produce agents useful for treating cirrhosis, these cells can be cultured in an appropriate medium under sterile conditions at 20-35° C. (e.g., 28-32° C.) for a sufficient amount of time (e.g., 60-150 hours) in an alternating electric field or a series of alternating electric fields as described above.

An exemplary set-up of the culture process is depicted in FIG. 1 (see above). An exemplary culture medium contains the following per 1000 ml of sterile water: 18 g of mannitol, 50 μg of Vitamin B₆, 80 μg of Vitamin B₁₂, 50 μg of Vitamin H, 100 [symbol] g of Vitamin E, 35 ml of fetal bovine serum, 0.2 g of KH₂PO₄, 0.25 g of MgSO₄.7H₂O, 0.3 g of NaCl, 0.2 g of CaSO₄.2H₂O, 4 g of CaCO₃.5H₂O, and 2.5 g of peptone. Yeast cells of the desired strain(s) are then added to the culture medium to form a mixture containing 1×10⁸ cells per 1000 ml of culture medium. The yeast cells can be of any of the strains listed in Table 1. The mixture is then added to the apparatus shown in FIG. 1.

The activation process of the yeast cells involves the following steps: (1) maintaining the temperature of the activation apparatus at 24-33° C. (e.g., 28-32° C.), and culturing the yeast cells for 24-30 hours (e.g., 28 hours); (2) applying an alternating electric field having a frequency of 7886 MHz and a field strength of 260-280 mV/cm (e.g., 274 mV/cm) for 11-17 hours (e.g., 15 hours); (3) then applying an alternating electric field having a frequency of 7907 MHz and a field strength of 300-330 mV/cm (e.g., 311 mV/cm) for 31-37 hours (e.g., 35 hours); (4) then applying an alternating electric field having a frequency of 12224 MHz and a field strength of 320-350 mV/cm (e.g., 337 mV/cm) for 39-45 hours (e.g., 43 hours); (5) then applying an alternating electric field having a frequency of 12646 MHz and a field strength of 340-370 mV/cm (e.g., 355 mV/cm) for 33-39 hours (e.g., 37 hours); and (6) then applying an alternating electric field having a frequency of 12662 MHz and a field strength of 270-290 mV/cm (e.g., 278 mV/cm) for 13-19 hours (e.g., 17 hours). The activated yeast cells are then recovered from the culture medium by various methods known in the art, dried (e.g., by lyophilization) and stored at 4° C. Preferably, the concentration of the dried yeast cells is no less than 10¹⁰ cells/g.

V. Acclimatization of Yeast Cells to the Gastric Environment

Because the yeast compositions of this invention must pass through the stomach before reaching the small intestine, where the effective components are released from these yeast cells, it is preferred that these yeast cells be cultured under acidic conditions to acclimatize the cells to the gastric juice. This acclimatization process results in better viability of the yeast cells in the acidic gastric environment.

To achieve this, the yeast powder containing activated yeast cells can be mixed with a highly acidic acclimatizing culture medium at 10 g (containing more than 10¹⁰ activated cells per gram) per 1000 ml. The yeast mixture is then cultured first in the presence of an alternating electric field having a frequency of 12646 MHz and a field strength of 350-380 mV/cm (e.g., 368 mV/cm) at about 28 to 32° C. for 40 to 50 hours (e.g., 45 hours). The resultant yeast cells can then be further incubated in the presence of an alternating electric field having a frequency of 12662 MHz and a field strength of 320-350 mV/cm (e.g., 324 mV/cm) at about 28 to 32° C. for 16 to 24 hours (e.g., 20 hours). The resulting acclimatized yeast cells are then dried and stored either in powder form (≧10¹⁰ cells/g) at room temperature or in vacuum at 0-4° C.

An exemplary acclimatizing culture medium is made by mixing 700 ml fresh pig gastric juice and 300 ml wild Chinese hawthorn extract. The pH of the acclimatizing culture medium is adjusted to 2.5 with 0.1 M hydrochloric acid (HCl) and 0.2 M potassium hydrogen phthalate (C₆H₄(COOK)COOH). The fresh pig gastric juice is prepared as follows. At about 4 months of age, newborn Holland white pigs are sacrificed, and the entire contents of their stomachs are retrieved and mixed with 2000 ml of water under sterile conditions. The mixture is then allowed to stand for 6 hours at 4° C. under sterile conditions to precipitate food debris. The supernatant is collected for use in the acclimatizing culture medium. To prepare the wild Chinese hawthorn extract, 500 g of fresh wild Chinese hawthorn is dried under sterile conditions to reduce water content (≦8%). The dried fruit is then ground (≧20 mesh) and added to 1500 ml of sterilized water. The hawthorn slurry is allowed to stand for 6 hours at 4° C. under sterile conditions. The hawthorn supernatant is collected to be used in the acclimatizing culture medium.

VI. Manufacture of Yeast Compositions

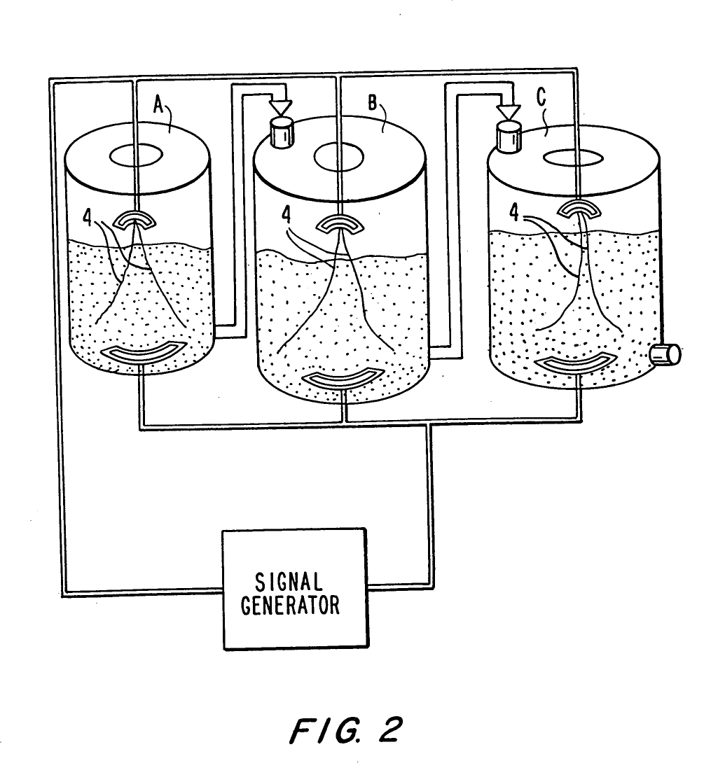

To manufacture the yeast compositions of the invention, an apparatus depicted in FIG. 2 or an equivalent thereof can be used. This apparatus includes three containers, a first container (A), a second container (B), and a third container (C), each equipped with a pair of electrodes (4). One of the electrodes is a metal plate placed on the bottom of the containers, and the other electrode comprises a plurality of electrode wires evenly distributed in the space within the container to achieve even distribution of the electric field energy. All three pairs of electrodes are connected to a common signal generator.

The culture medium used for this purpose is a mixed fruit extract solution containing the following ingredients per 1000 L: 300 L of wild Chinese hawthorn extract, 300 L of jujube extract, 300 L of Schisandra chinensis (Turez) Baill seed extract, and 100 L of soy bean extract. To prepare hawthorn, jujube and Schisandra chinensis (Turez) Baill seed extracts, the fresh fruits are washed and dried under sterile conditions to reduce the water content to no higher than 8%. One hundred kilograms of the dried fruits are then ground (≧20 mesh) and added to 400 L of sterilized water. The mixtures are stirred under sterile conditions at room temperature for twelve hours, and then centrifuged at 1000 rpm to remove insoluble residues. To make the soy bean extract, fresh soy beans are washed and dried under sterile conditions to reduce the water content to no higher than 8%. Thirty kilograms of dried soy beans are then ground into particles of no smaller than 20 mesh, and added to 130 L of sterilized water. The mixture is stirred under sterile conditions at room temperature for twelve hours and centrifuged at 1000 rpm to remove insoluble residues. To make the culture medium, these ingredients are mixed according to the above recipe, and the mixture is autoclaved at 121° C. for 30 minutes and cooled to below 40° C. before use.

One thousand grams of the activated yeast powder prepared as described above (Section V, supra) is added to 1000 L of the mixed fruit extract solution, and the yeast solution is transferred to the first container (A) shown in FIG. 2. The yeast cells are then cultured in the presence of an alternating electric field having a frequency of 12646 MHz and a field strength of about 400-440 mV/cm (e.g., 413 mV/cm) at 28-32° C. under sterile conditions for 32 hours. The yeast cells are further incubated in an alternating electric field having a frequency of 12662 MHz and a field strength of 330-370 mV/cm (e.g., 347 mV/cm). The culturing continues for another 12 hours.

The yeast culture is then transferred from the first container (A) to the second container (B) which contains 1000 L of culture medium (if need be, a new batch of yeast culture can be started in the now available first container (A)), and subjected to an alternating electric field having a frequency of 12646 MHz and a field strength of 430-470 mV/cm (e.g., 442 mV/cm) for 24 hours. Subsequently the frequency and field strength of the electric field are changed to 12662 MHz and 350-380 mV/cm (e.g., 364 mV/cm), respectively. The culturing continues for another 12 hours.

The yeast culture is then transferred from the second container (B) to the third container (C) which contains 1000 L of culture medium, and subjected to an alternating electric field having a frequency of 12646 MHz and a field strength of 310-340 mV/cm (e.g., 324 mV/cm) for 24 hours. Subsequently the frequency and field strength of the electric field are changed to 12662 MHz and 260-280 mV/cm (e.g., 274 mV/cm), respectively. The culturing continues for another 12 hours.

The yeast culture from the third container (C) can then be packaged into vacuum sealed bottles for use as dietary supplements, e.g., health drinks, or medication in the form of pills, powder, etc. If desired, the final yeast culture can also be dried within 24 hours and stored in powder form. The dietary supplement can be taken three to four times daily at 30-60 ml per dose for a three-month period, preferably 10-30 minutes before meals and at bedtime.

In some embodiments, the compositions of the invention can also be administered intravenously or peritoneally in the form of a sterile injectable preparation. Such a sterile preparation can be prepared as follows. A sterilized health drink composition is first treated under ultrasound (20,000 Hz) for 10 minutes and then centrifuged for another 10 minutes. The resulting supernatant is adjusted to pH 7.2-7.4 using 1 M NaOH and subsequently filtered through a membrane (0.22 μm for intravenous injection and 0.45 μm for peritoneal injection) under sterile conditions. The resulting sterile preparation is submerged in a 35-38° C. water bath for 30 minutes before use. In other embodiments, the compositions of the invention may also be formulated with pharmaceutically acceptable carriers to be orally administered in any orally acceptable dosage form including, but not limited to, capsules, tablets, suspensions or solutions.

The yeast compositions of the present invention are derived from yeasts used in food and pharmaceutical industries. The yeast compositions are thus devoid of side effects associated with many pharmaceutical compounds.

### VII. EXAMPLES

The following examples are meant to illustrate the methods and materials of the present invention. Suitable modifications and adaptations of the described conditions and parameters which are obvious to those skilled in the art are within the spirit and scope of the present invention.

The activated yeast compositions used in the following experiments were prepared as described above, using Saccharomyces cerevisiae Hansen AS2.562 cells cultured in the presence of an alternating electric field having the electric field frequency and field strength exemplified in the parentheses following the recommended ranges listed in Section IV, supra. Control yeast compositions were those prepared in the same manner except that the yeast cells were cultured in the absence of EMFs. Unless otherwise indicated, the yeast compositions and the corresponding controls were administered to the animals by intragastric feeding.

### Example 1

### Effects of Yeast Compositions on Fibrous Tissue Formation and Collagen Level in Liver

Fibrous tissue formation as a result of liver cell regeneration and high collagen level are characteristics of liver cirrhosis. To test the ability of the yeast composition containing EMF-treated AS2.562 cells to ameliorate or prevent cirrhosis, the composition's effects on liver fibrous tissue formation and collagen level were examined in Wistar rats with liver cirrhosis induced by subcutaneous injection of CCl₄. The activated yeast composition of this invention was shown to significantly alleviate these symptoms of cirrhosis. This result was obtained as follows.

Forty Wistar rats (half male, half female, 6-9 months old, and 250-280 g in weight) were divided randomly into four groups of ten rats each: AY, for treatment with activated yeast composition; NY, for treatment with control yeast composition (unactivated yeast); CK1, control group for treatment with saline; and CK2, normal control without induction of cirrhosis for treatment with saline.

To induce cirrhosis, on day one of the nine-week experiment the AY, NY, and CK1 groups of rats were each administered 5.0 ml/kg (body weight) CCl₄ by subcutaneous injection. Each rat was then injected with 3 ml/kg of CCl₄ containing 40% plant oil (such as peanut oil). For the first two weeks, the rats' diet contained 79.5% corn flour, 20% lard, and 0.5% cholesterol, and their drinking water contained 30% alcohol. From the third week to the end of the ninth week, the diet contained 99% corn flour and 1% cholesterol, and the drinking water contained 30% alcohol.

Starting from the second day of the experiment, each AY rat was administered 1.5 ml per 100 g body weight of the activated yeast composition twice daily till the end of the experiment; rats in groups NY and CK1 were given the control yeast composition and saline at the same dosage, respectively. The fourth group of rats, CK2, were not challenged with CCl₄ but were fed normally and provided normal drinking water during the nine-week period. They were given 1.5 ml of saline twice daily starting from the second day of the experiment. The four groups of rats were otherwise maintained under the same conditions.

At the end of the ninth week, each rat was sacrificed and the left lobe of the liver was fixed in 10% formaldehyde. Paraffin sections were prepared and stained with HE (hematoxylin-eosin) and/or VG (van Gieson), and fibrous tissue formation was examined under the microscope. The rest of the liver sample was immersed first in 95% ethanol for 12 hours and then in acetone for 48 hours to extract fat. The liver was then dried at 110° C. and ground into powder.

To measure the liver hydroxyproline (“Hyp”) level, 40 mg of the liver powder was added to 3 ml of 6 M HCl and incubated at 125° C. to hydrolyze for five hours. The sample was then cooled down to room temperature and its pH adjusted to 6.0 with 6 M NaOH. The volume was brought up to 50 ml with de-ionized water. After filtration, 2 ml of the resulting solution was mixed with 1 ml of chloramine-T and incubated at room temperature for twenty minutes. One milliliter of perchloric acid was subsequently added. Five minutes later, 1 ml of 10% p-dimethylaminobenzaldehyde was added and the reaction was incubated in a 60° C. water bath for 20 minutes for color to develop. Optical densities of the samples were then measured at 550 nm. Hyp levels (Y) of the samples were obtained based on a proline standard curve. The proline standard curve was made by assaying proline solutions of several different concentrations following the procedure as described above. Since every microgram (μg) of Hyp corresponds to about 7.46 microgram (μg) of collagen in the liver, the liver collagen level (X) was calculated by the following formula:
X=[(7.46×50)/40]×Y=9.325×Y (mg per gram liver dry weight).

The data from the above experiments are summarized in Table 2 below.

TABLE 2

| | | Fibrous tissue formation in liver | | | | | Collagen |
|---|---|---|---|---|---|---|---|
| | # | | | | | Average | (mg/g dry |
| Group | rats | −* | + | ++ | +++ | (%)** | liver) |
| AY | 10 | 8 | 2 | 0 | 0 | 0.4 | 16.7 ± 6.2 |
| NY | 10 | 0 | 0 | 3 | 7 | 2.9 | 37.8 ± 18.3 |
| CK1 | 10 | 0 | 0 | 2 | 8 | 3.1 | 38.6 ± 17.4 |
| CK2 | 10 | 10 | 0 | 0 | 0 | 0 | 15.3 ± 5.5 |

*“-”: no fibrous tissue; “+”: 0-0.25%, fibrous tissue volume v. total liver volume;
“++”: 0.25-2.5%; “+++”: 2.5-5.0%.
**Average fibrous tissue volume as percent of total liver volume.

As shown in Table 2 above, the CK1 rats developed severe cirrhosis, indicating the success of cirrhosis induction by CCl₄. The AY rats, like the healthy control CK2 rats, had significantly less fibrous tissue formation or collagen in the liver compared to CK1 rats, while the NY rats were similar to CK1 rats in terms of the severity of cirrhosis. These data demonstrate that the activated yeast composition can significantly alleviate the symptoms of liver cirrhosis, e.g., decrease liver collagen level and the formation of liver fibrous tissue, as compared to the control yeast composition.

### Example 2

### Effects of Yeast Compositions on the Serum γ-Globulin Level

Serum proteins are generally classified into albumin and globulins. Globulins are roughly divided into α, β, and γ globulins, which can be separated and quantitated by electrophoresis and densitometry. The γ-globulins include the various types of antibodies, such as immunoglobulins M, G, and A. When the liver tissue is damaged as in cirrhosis, serum γ-globulin levels increase because B cells secret more antibodies as a result of, inter alia, the saturated phagocytosis capability of the Kuffer cells and inadequate T-cell function. Thus, serum γ-globulin level is one of the important indicators of liver functions.

To evaluate the effects of the activated yeast composition of this invention on serum γ-globulin levels, rats with CCl₄-induced liver cirrhosis were treated with the yeast compositions according to the procedure described in Example 1. The rats were sacrificed at the end of the ninth week. Blood samples were drawn from each of the sacrificed rats and sera were prepared. To determine the relative serum y-globulin level, the sera were subjected to standard serum globulin electrophoresis. After the electrophoresis was completed, the electrophoresis membrane was stained in amido black 10 B solution for 10 minutes, and then destained to get rid of background staining. Each of the albumin or globulin bands was then excised. The membrane containing albumin was soaked in 6 ml of 0.4 M NaOH in a test tube, and the globulin bands were each soaked in 3 ml of 0.4 NaOH. All tubes were incubated at room temperature for an hour with agitation to elute the dye from the membrane. The optical density of each sample was measured at 580 nm, using 0.4 M NaOH for calibration. The relative proportion of each protein fraction was calculated using the following formulae:
Total serum protein=ΣE=2×E_{A}*+E_{α1}+E_{α2}+E_{β}+E_{γ}
albumin (%)=[(2×E_{A})/ΣE]×100
α1 globulin (%)=(E_{α1}/ΣE)×100
α2 globulin (%)=(E_{α2}/ΣE)×100
β globulin (%)=(E_{β}/ΣE)×100
γ globulin (%)=(E_{γ}/ΣE)×100

*E: optical density; A: albumin.

The average serum y-globulin level (as percent of total serum protein) for the different groups of rats were shown in Table 3 below.

TABLE 3

| | | | γ-globulin |
|---|---|---|---|
| Group | # rats | Treatment | level (%) |
| AY | 10 | cirrhosis rat with activated | 13.9 ± 2.1 |
| | | yeast comp | |
| NY | 10 | cirrhosis rat with control | 25.9 ± 4.3 |
| | | yeast comp | |
| CK1 | 10 | cirrhosis rat with saline | 26.6 ± 4.5 |
| CK2 | 10 | healthy rat with saline | 15.7 ± 3.3 |

The data demonstrate that the activated yeast composition was effective in maintaining normal serum γ-globulin levels in rats with cirrhosis, while the control yeast composition was not.

While a number of embodiments of this invention have been set forth, it is apparent that the basic constructions may be altered to provide other embodiments which utilize the compositions and methods of this invention.

1. A composition comprising a plurality of yeast cells, wherein said plurality of yeast cells are characterized by their ability to decrease liver collagen level or formation of liver fibrous tissue or to normalize serum γ-globulin level in a subject, said ability resulting from their having been cultured in the presence of an alternating electric field having a frequency in the range of 7700-12800 MHz and a field strength in the range of 240-500 mV/cm, as compared to yeast cells not having been so cultured.

2. The composition of claim 1, wherein said frequency is in the range of 7800-8000, 12150-12300, or 12550-12800 MHz.

3. The composition of claim 1, wherein said field strength is in the range of 260-280, 270-290, 300-330, 310-340, 320-350, 330-370, 340-370, 350-380, 400-440, or 430-470 mV/cm.

4. The composition of claim 1, wherein said yeast cells are of the species selected from the group consisting of Saccharomyces cerevisiae, Saccharomyces carlsbergensis, Saccharomyces rouxii, Saccharomyces sake, Saccharomyces uvarum, Saccharomyces sp., Schizosaccharomyces pombe, and Rhodotorula aurantiaca.

5. The composition of claim 1, wherein said yeast cells are of the strain deposited at the China General Microbiological Culture Collection Center with an accession number selected from the group consisting of Saccharomyces cerevisiae Hansen AS2.562 and AS2.69, Saccharomyces sp. AS2.311, Schizosaccharomyces pombe Lindner AS2.994, Saccharomyces sake Yabe ACCC2045, Saccharomyces uvarum Beijer IFFI1044, Saccharomyces rouxii Boutroux AS2.180, Saccharomyces cerevisiae Hansen Var. ellipsoideus AS2.612, Saccharomyces carlsbergensis Hansen AS2.377, or Rhodotorula rubar (Demme) Lodder AS2.282.

6. The composition of claim 1, wherein said composition is in the form of a tablet, powder, or a health drink.

7. The composition of claim 1, wherein said composition is in the form of a health drink.

8. A method of treating liver cirrhosis in a subject, comprising administering the composition of claim 1 to the subject.

9. The method of claim 8 comprising oral administration.

10. A method of preparing a yeast composition, comprising culturing a plurality of yeast cells in the presence of an alternating electric field having a frequency in the range of 7700-12800 MHz and a field strength in the range of 240-500 mV/cm for a period of time sufficient to substantially increase the capability of said plurality of yeast cells to decrease liver collagen level or formation of liver fibrous tissue or to normalize serum γ-globulin level.
